(19) United States
(12) Patent Application Publication
(10) Pub. No.: US 2003/0232741 A1
(43) Pub. Date: Dec. 18, 2003

(54) Methods of treatment of glaucoma and other conditions mediated by NOS-2 expression via inhibition of the EGFR pathway

(75) Inventors: Arthur H. Neufeld, St. Louis, MO (US); Bin Liu, St. Louis, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Washington University

(21) Appl. No.: 10/430,527

(22) Filed: May 6, 2003

Related U.S. Application Data
(60) Provisional application No. 60/378,254, filed on May 6, 2002.

(51) Int. Cl. A61K 31/00

(52) U.S. Cl. 514/1

(57) ABSTRACT

Therapeutic methods and compositions for the treatment of glaucoma and other conditions mediated at least in part by the expression of NOS-2 are provided.

This application claims priority to U.S. provisional patent application Serial No. 60/378,254, filed May 6, 2002. The text of that application is hereby incorporated by reference.

This invention was made with United States Government support awarded by the National Institute of Health (NIH), Grant No. EY-12017. The United States Government has certain rights in this invention.

### FIELD OF THE INVENTION

This invention discloses a previously unknown signal transduction pathway leading from the activation of EGFR to an increase in NOS-2 activity and provides therapeutic methods and compositions related to this discovery. Accordingly, the invention relates to compositions and methods for the treatment and prevention of conditions in which excessive nitric oxide produced by NOS-2 resulting from activation of the EGFR pathway are implicated. This invention also relates, in particular, to the treatment of ocular disorders of the eye, and more particularly, to the treatment of glaucomatous optic neuropathy, and related conditions, such as primary and secondary glaucoma, normal pressure glaucoma, and ocular hypertension, through the use of signal transduction inhibitors of the above mentioned signaling pathway.

### BACKGROUND OF THE INVENTION

Glaucoma, the second leading cause of irreversible loss of vision in the world, is characterized by loss of visual field due to optic nerve degeneration, usually in response to abnormally elevated intraocular pressure. In glaucomatous optic neuropathy, the initial site of neuronal damage is at the level of the lamina cribrosa of the optic nerve head (ONH).^{1,2} Within this region, the axons of the retinal ganglion cells degenerate and the supporting connective tissue undergoes extensive remodeling.³ Astrocytes, the major glial cell type in the nonmyelinated ONH in humans, become reactive astrocytes and markedly change their morphology, distribution and function as the chronic glaucomatous process proceeds.⁴ The local cellular responses of these reactive astrocytes alter the microenvironment of the axons of the retinal ganglion cells and may contribute primarily or secondarily to the axonal damage.

Applicants have demonstrated the induction of nitric oxide synthase-2 (NOS-2), also known as inducible nitric oxide synthase (iNOS), in reactive astrocytes of the optic nerve heads of patients with primary open angle glaucoma and have provided evidence suggesting that excessive nitric oxide, produced by NOS-2, causes local neurotoxicity and degeneration of the axons of the retinal ganglion cells in the glaucomatous ONH.^{5,6} Applicants' pharmacological experiments have proven that the pathological expression of NOS-2 in the ONH causes the loss of retinal ganglion cells in a rat model of glaucoma associated with chronic, moderately elevated intraocular pressure.⁷ Applicants' in vitro experiments have demonstrated that the expression of NOS-2 in astrocytes of the human ONH can be induced by elevated hydrostatic pressure.⁸

The intracellular pathway that mediates the induction of NOS-2 in response to elevated pressure in human glaucomatous optic nerve astrocytes is unknown. Communication through cell surface molecules and downstream cellular signaling pathways have emerged as common principles that enable cells to integrate a multitude of signals from the environment. One major family of sensors is comprised of transmembrane receptors with intrinsic protein tyrosine kinase activity. The prototypal member is the epidermal growth factor receptor (EGFR), also referred to as HER (human EGF receptor) and c-erbB1.⁹ Stimulation of EGFR causes activation of the receptor tyrosine kinase activity, autophosphorylation, internalization of the ligand-EGFR complex, translocation to the nucleus and, eventually, degradation in lysosomes.^{10,11} The integrated biological responses to EGFR stimulation control basic cell functions such as mitogenesis, apoptosis, enhanced cell mobility, protein secretion, and differentiation or de-differentiation.⁹

Several types of inhibitors of EGFR activity have been reported. Some such inhibitors are structurally unrelated to EGF or EGFR, such as cyclosporin A, interferon-γ, chrysarobin and TGF-α.¹²⁻¹³ Prostaglandin and some anti-EGFR monoclonal antibodies and phorbol esters also are known to inhibit stimulation of certain target cells by EGF.¹³⁻¹⁶ Several monoclonal anti-EGFR antibodies inhibit EGF-dependent growth of a human breast carcinoma cell line in vitro.¹⁷

EGF-like proteins and peptides have also been used to inhibit growth stimulation of target cells by EGF. Small proteins that compete with EGF for EGFR, and mimic EGF activity on target cells have been identified in two human tumors.¹⁸ Engineered mutants of EGF are associated with decreased EGF-stimulated tyrosine kinase activity.¹⁹ It has been reported that a synthetic peptide encompassing the third disulfide loop of TGF-α inhibits EGFR-related growth of human mammary carcinoma cells, although proliferation stimulated by fibroblasts or platelet derived growth factors was unaltered.²⁰

Applicants demonstrate that in vivo, EGFR and phosphorylated EGFR are abundantly present in astrocytes of the ONHs from patients with primary open-angle glaucoma, and in vitro, phosphorylation of EGFR is markedly enhanced in the nucleus of human ONH astrocytes in response to elevated hydrostatic pressure. As detailed below, identification of this crucial intracellular pathway that leads to neurotoxicity in glaucomatous optic neuropathy enables new approaches to pharmacological neuroprotection for the treatment of glaucoma.

Additionally, Nitric Oxide Synthase-2 (NOS-2) has been implicated in a number of neurodegenerative diseases, including stroke, Parkinson's disease, Amyotrophic Lateral Sclerosis (Lou Gehrig's disease), Alzheimer's disease and multiple sclerosis. Accordingly, as discussed below, methods of pharmacological neuroprotection therapy are provided for neurodegenerative conditions mediated at least in part by NOS-2 based on the pharmacological inhibition of EGFR, given EGFR's role in the induction of NOS-2.

### SUMMARY OF THE INVENTION

Applicants have discovered that by inhibiting the EGFR pathway, that induction of NOS-2 can be inhibited, thereby providing a therapeutic route for treatment or prevention of conditions in which excessive nitric oxide produced by NOS-2 are implicated.

In accordance with the invention, applicants have provided a method of inhibiting expression of NOS-2 in a subject in need of such inhibition. The method comprises administering to the subject an effective amount of an inhibitor of the EGFR pathway. In preferred embodiments, the inhibitor is a specific inhibitor of EGFR's tyrosine kinase activity or an inhibitor which binds directly or indirectly to EGFR, e.g., an antibody. In practice, the inhibitor is used to treat or prevent a condition mediated at least in part by the expression of NOS-2. Accordingly, applicants provide methods and compositions which treat or prevent neurological conditions resulting at least in part from EGFR pathway-mediated induction of NOS-2 expression in astrocytes. In particular, conditions subject to treatment as a result of applicants' discoveries include neurological disorders or neurodegenerative diseases, including Alzheimer's disease, Parkinson's disease, Amyotrophic Lateral Sclerosis (Lou Gehrig's disease), multiple sclerosis, motor-neuron disease, diabetic retinopathy, glaucomatous optic neuropathy, myasthenia gravis, tardine dyskinesia, dementia associated with Down's syndrome, stroke, cerebral ischemia, senile cognitive decline, a demyelinating condition or mechanical injury.

In another embodiment, the condition is selected from the group consisting of a degenerative bone disease, an inflammatory disease, or a condition caused by compression of a tissue leading to damage, such as arthritis, osteoarthritis, and rheumatoid arthritis.

Also provided is a method of inhibiting the activity of a pressure-sensitive promoter region of a gene in a subject through the administration of an inhibitor selected from the group consisting of an inhibitor of EGFR's tyrosine kinase activity or an inhibitor which binds directly to EGFR. In this method, the inhibitor is selective in that it does not substantially inhibit the promoter's activity leading to mRNA or protein expression which is induced by an inflammatory cytokine. In one embodiment, the pressure-sensitive promoter is the human NOS-2 promoter.

In a further embodiment of the invention, a method for treating or preventing damage associated with glaucoma is provided. In the method, an effective amount of an inhibitor of NOS-2 expression is administered to the eye of a subject in need thereof. In preferred embodiments, the inhibitor is an inhibitor of EGFR's tyrosine kinase activity or an inhibitor which binds directly to EGFR, e.g., an antibody or antagonist.

Another aspect of the invention relates to the provision of pharmaceutical compositions useful for carrying out the disclosed therapeutic methods. In this embodiment, the pharmaceutical composition comprises an amount of an inhibitor of the EGFR pathway or an appropriate precursor, prodrug, metabolite, analog or derivative thereof, in applicable dosage units for the condition, effective to inhibit expression of NOS-2. The composition also includes a pharmaceutically acceptable carrier.

In addition, the invention relates to methods for identifying therapeutics useful for the prevention and/or treatment of glaucoma and other conditions in which NOS-2 is implicated.

Other objects and advantages of the present invention will become apparent as the detailed description of the invention proceeds.

### BRIEF DESCRIPTION OF THE FIGURES

These and other objects, features and many of the attendant advantages of the invention will be better understood upon a reading of the following detailed description when considered in connection with the accompanying drawings wherein:

FIG. 1.

Immunohistochemistry for EGFR and p-EGFR in human normal and glaucomatous ONHs

a, Normal ONHs have very few cells positive for EGFR. b, Glaucomatous ONHs have many cells positive for EGFR (arrows). c-h, Staining for p-EGFR (arrowheads) in glaucomatous ONHs. c, White arrowheads show co-localization of p-EGFR and GFAP in astrocytes. d, Labeling for p-EGFR in the cytoplasm and nucleus of astrocytes in disorganized area of the glaucomatous ONH. In glaucomatous ONHs, positively labeled astrocytes for p-EGFR are abundantly present in disorganized areas in the prelaminar region (e), in the lamina cribrosa region (f, and in the postlaminar region (g), but are less frequent in nearby areas with relatively normal structure (h). NB, nerve bundle; dNB, damaged nerve bundle; CP, cribriform plates. Magnifications: a, b, c, ×600; d, ×1000; e, f, g, h, ×400.

FIG. 2.

Detection of EGFR phosphorylation in response to elevated hydrostatic pressure in cultured human ONH astrocytes

a-f, Immunocytochemistry for EGFR (a, b) and p-EGFR (c-f). a, b, c, control astrocytes. d, e, astrocytes exposed to elevated hydrostatic pressure for 10 min. Note the enhanced labeling for p-EGFR in the nucleus and the specific filamentous appearance of p-EGFR labeling in the cytoplasm of astrocytes exposed to elevated hydrostatic pressure for 10 min. f, Treatment with AG82 before and during the period of elevated hydrostatic pressure prevents the nuclear labeling for p-EGFR. Magnifications: a, c, d, f, ×600; b, e, ×1000. g, Immunoblot for EGFR and p-EGFR. Note the increase in p-EGFR in both nuclear and non-nuclear fractions within 10 min of exposure to elevated hydrostatic pressure. Treatment with AG82 completely prevents the increase in p-EGFR in both nuclear and non-nuclear fractions following exposure to elevated hydrostatic pressure. EGFR appears unchanged in response to elevated hydrostatic pressure. GFAP is the loading control.

FIG. 3.

Tyrosine kinase inhibitors block NOS-2 induction by elevated hydrostatic pressure in astrocytes

a, b, Treatment with AG82. c, d, Treatment with AG18. NOS-2 expression is detected at the protein level by Western blot (a, c) and at the mRNA level by semi-quantitative RT-PCR (b, d). AG82 and AG18 have both significantly blocked the appearances of NOS-2 mRNA and protein.

FIG. 4.

EGFR ligand dependent induction of NOS-2 by elevated hydrostatic pressure

a, Immunoblot for NOS-2. Pre-incubation with EGFR antibody significantly blocks NOS-2 induction by elevated hydrostatic pressure and EGF. (b, c) Immunocytochemistry for NOS-2 and GFAP. Compared with control astrocytes (b), astrocytes incubated with EGF for 48 hrs (c) are elongated and have increased labeling for NOS-2 and GFAP. Magnification: ×600.

FIG. 5.

NFκB Inhibitors block NOS-2 induction in response to cytokines and elevated hydrostatic pressure

FIG. 5 depicts the effects of an inhibitor of NFκB, SN50, on cytokine induction and pressure sensitive induction of NOS-2. (a) Immunoblot for NOS-2 protein. (b) Mean ±SEM of gel density scans of several immunoblots normalized to the values in the control lanes. (c) RT-PCR of mRNA for NOS-2. Beta-actin is the internal loading control.

FIG. 6.

MAP Kinase Inhibitors block NOS-2 induction in response to cytokines, but not to elevated hydrostatic pressure

FIG. 6 depicts the effects of an inhibitor of MAP kinase, SB202190, on cytokine induction and pressure induction of NOS-2. (a) Immunoblot for NOS-2 protein. (b) Mean ±SEM of gel density scans of several immunoblots normalized to the values in the control lanes. (c) RT-PCR of mRNA for NOS-2. Beta-actin is the internal loading control.

FIG. 7.

Protein Tyrosine Kinase inhibitors block NOS-2 induction in response to elevated hydrostatic pressure, but not to cytokines

FIG. 7 depicts the effects of an inhibitor of protein tyrosine kinase, AG82, on cytokine induction and pressure induction of NOS-2. (a) Immunoblot for NOS-2 protein. (b) Mean ±SEM of gel density scans of several immunoblots normalized to the values in the control lanes. (c) RT-PCR of mRNA for NOS-2. Beta-actin is the internal loading control.

### DEFINITIONS

To facilitate understanding of the invention, a number of terms are defined below. Definitions of certain terms are included here. Any term not defined is understood to have the normal meaning used by scientists contemporaneous with the submission of this application.

The term “effective amount” refers to that amount of a preparation that, when administered to a particular subject in view of the nature and severity of that subject's disease or condition, will have the desired effect, e.g., an amount which will cure, or at least partially arrest or inhibit the disease or condition. For example, an “effective amount” may be that required to successfully treat glaucoma. The effective amount may depend on a number of factors, including the age, race, and sex of the subject and the severity of the glaucoma and other factors responsible for biologic variability. Though the term effective amount is not limited to a particular mechanism of action for a specific compound, an effective amount may be that amount of a compound able to inhibit the phosphorylation of cell surface and intracellular EGFR by natural and non-natural ligand-dependent mechanisms or by ligand-independent mechanisms.

The term “glaucoma” refers to an ophthalmologic disorder responsible for visual impairment. The disease is characterized by a progressive neuropathy caused at least in part by deleterious effects resulting from intraocular pressure on the optic nerve. The term glaucoma refers broadly to both primary glaucomas, which include normal pressure glaucomas, open-angle, angle-closure, and congenital glaucomas, and secondary glaucomas, which occur as a sequel to ocular injury or preexisting disease, as well as ocular hypertension, which can occur before glaucomatous optic neuropathy. Though not limited to any particular type of glaucoma, it is anticipated that the pharmacological agents and compounds of the present invention will be most efficacious in the treatment of primary glaucoma.

### DETAILED DESCRIPTION OF THE INVENTION

The present invention relates to the treatment of neurodegenerative diseases such as glaucoma and other conditions mediated at least in part by the expression of NOS-2. While the present invention does not depend on an understanding of the mechanism by which successful treatment is accomplished, it is believed that the therapeutic method of the present invention inhibits the EGFR tyrosine kinase pathway which is a necessary component for signal transduction to induce NOS-2 in response to elevated pressure in cells and tissues such as the astrocytes of glaucomatous human ONH.

The present invention, therefore, is directed towards methods and compositions which utilize cellular signal transduction inhibitors that serve to prevent and/or treat a subject with conditions such as glaucoma.

In a particular embodiment, the present invention contemplates a method of treating or preventing glaucoma, comprising (a) providing a mammal with, or at risk of contracting, glaucoma; and (b) administering to the mammal an effective amount of a non-toxic inhibitor of EGFR tyrosine kinase activity, thereby inhibiting optic nerve degeneration.

A variety of inhibitors of EGFR have been identified, including a number already undergoing clinical trials for treatment of various cancers. For a recent summary, see de Bono, J. S. and Rowinsky, E. K. (2002), “The ErbB Receptor Family: A Therapeutic Target For Cancer”, Trends in Molecular Medicine, 8, S19-26. Identified inhibitors include antibodies (see, e.g. Wels , et al., U.S. Pat. No. 6,129,915 and Careller, et al., U.S. Pat. No. 5,969,107), antisense and related oligonucleotides (Wyatt, et al., U.S. Pat. No. 6,444,465) and natural (e.g., lavendustin A) and synthetic (e.g., ZD 1839) EGFR protein tyrosine kinase inhibitors. Those identified by de Bono and Rowinsky as having advanced to clinical evaluation include: The quinazoline EGFR inhibitors ZD1839 (Iressa®, gifitinib; AstraZeneca, London, U.K.); OSI-774 (Tarceva®; OSI Pharmaceuticals, Uniondale, N.Y.); GW2016 (GlaxoSmithKline, London, U.K.); and Cl-1033 (PD183805, Pfizer, New York, N.Y.); a pyrolopyrimidine, PK1 116 (Novartis, Basel, SW.); and a 3-Cyanoquinoline, EKB-569 (Wyeth, Madison, N.J.); antibodies, including (IMC-C225 (Cetuximab®, Imclone Systems, New York, N.Y.), a chimeric mAb directed against the extra-cellular domain of the EGF receptor; ABX-EGF (Abgenix, Fremont, Calif.), a fully humanized monoclonal antibody specific to EGFR and MDX-447 (Medarex, Princeton, N.J.), a bispecific antibody specific for EGFR and the IgG receptor CD64; and conjugates of toxins to EGF ligands and immunoconjugates (e.g., anti-EGFR mAbs IMC-C225 and -528 with ricin A chain). ZD1839 has received FDA approval for use in lung cancer treatment. For additional types of EGFR inhibitors, see Nadel, et al., U.S. Pat. No. 6,551,989 and references listed therein.

Examples of EGFR tyrosine kinase inhibitors which may potentially be utilized in the invention are set forth in Table 1.

TABLE 1
Epidermal growth factor receptor tyrosine kinase inhibitors

| | Common Name/ | |
|---|---|---|
| Compound | Trade Name | Supplier |
| 4-(3-Chloro-4- | ZD1839, gefitinib | AstraZeneca, London, UK |
| fluorophenylamine)-7- | | |
| methoxy-6-(3-(4- | | |
| morpholinyl)guinazoline | | |
| | Cl-1033 | Parke-Davis & Co.; Pfizer |
| | Erbitux (C225); Cetuximab | BristolMyersSquibb; |
| | | Imclone |
| | ABX-EGF | Abgenix, Fremont, CA. |
| | GW572016 | GlaxoSmithKline |
| | PKI 166 | |
| (+)-Aeroplysinin-1, | | CAS 28656-91-9; |
| Aplysina aerophoba | | Calbiochem, San Diego, |
| | | CA |
| 2-Naphthylvinyl ketone | JAK3 Inhibitor V | Calbiochem, San Diego, |
| | | CA |
| (E)-3-(3,5-Diisopropyl-4- | SU1498 | Calbiochem, San Diego, |
| hydroxyphenyl)-2-[(3- | | CA |
| phenyl-n-propyl)amino- | | |
| carbonyl]acrylonitrile | | |
| α-(3′-Pyridyl)-(3,5- | RG-14620 | Calbiochem, San Diego, |
| dichloro)cinnamonitrile | | CA |
| α-Cyano-(+)-(S)-N-(a- | AG82 | Calbiochem, San Diego, |
| phenethyl)-(3,4- | | CA. |
| dihydroxy)cinnamide | | |
| α-Cyano-(3,4,5- | | |
| trihydroxy)cinnamonitrile | | |
| α-Cyano-(3,4-dihydroxy)- | AG555 | Calbiochem, San Diego, |
| N-(3- | | CA |
| phenylpropyl)cinnamide | | |
| α-Cyano-(3,4-dihydroxy)- | AG556 | Calbiochem, San Diego, |
| N-(4- | | CA |
| phenylbutyl)cinnamide | | |
| α-Cyano-(3,4-dihydroxy)- | AG490 | Calbiochem, San Diego, |
| N-benzylcinnamide | | CA |
| α-Cyano-(3,4-dihydroxy)- | AG494 | Calbiochem, San Diego, |
| N-phenylcinnamide | | CA |
| α-Cyano-(3,4- | AG99 | Calbiochem, San Diego, |
| dihydroxy)cinnamide | | CA |
| α-Cyano-(3,4- | AG18 | Calbiochem, San Diego, |
| dihydroxy)cinnamonitrile | | CA |
| α-Cyano-(3,4- | AG213 | Calbiochem, San Diego, |
| dihydroxy)thiocinnamide | | CA |
| α-Cyano-b-hydroxy-b- | LFM-A12 | Calbiochem, San Diego, |
| methyl-N-[4- | | CA |
| (trifluoromethoxy)phenyl] | | |
| propenamide | | |
| 2′,4′,3,4- | Butein | Sigma-Aldrich Co., St. |
| Tetrahydroxychalcone | | Louis, MO |
| 2,5- | Erbstatin Analog | Calbiochem, San Diego, |
| Dihydroxymethylcinnamate | | CA |
| 2-Amino-4-(1H-indol-5-yl)- | AG370 | Calbiochem, San Diego, |
| 1,1,3-tricyanobuta-1,3- | | CA |
| diene | | |
| 2-Amino-4-(3′,4′,5′- | AG183 | Calbiochem, San Diego, |
| trihydroxyphenyl)-1,1,3- | | CA |
| tricyanobuta-1,3-diene | | |
| 2-Amino-4-(4′- | AG122 | Calbiochem, San Diego, |
| hydroxyphenyl)-1,1,3- | | CA |
| tricyanobuta-1,3-diene | | |
| 2′-Thioadenosine | PD 157432 | Calbiochem, San Diego, |
| | | CA |
| 3-Hydroxy-1- | Damnacanthal | Calbiochem, San Diego, |
| methoxyanthraquinone-2- | | CA |
| aldehyde | | |
| 4-(3′,5′-Dibromo-4- | JAK3 Inhibitor III | Calbiochem, San Diego, |
| hydroxyphenyl)amino-6,7- | | CA |
| dimethoxyquinazoline | | |
| 4-(3-Chloroanilino)-6,7- | AG 1478, PD153035 | Calbiochem, San Diego, |
| dimethoxyquinazoline | | CA |
| 4-[(3- | BPDQ | Calbiochem, San Diego, |
| Bromophenyl)amino]-6,7- | | CA |
| diaminoquinazoline | | |
| 4-[(3- | Compound 56 | Calbiochem, San Diego, |
| Bromophenyl)amino]-6,7- | | CA |
| diethoxyquinazoline | | |
| 4-[(3- | PD 158780 | Calbiochem, San Diego, |
| Bromophenyl)amino]-6- | | CA |
| (methylamino)-pyrido[3,4- | | |
| d]pyridimine | | |
| 4-[(3- | PD 168393 | Calbiochem, San Diego, |
| Bromophenyl)amino]-6- | | CA |
| acrylamidoquinazoline | | |
| 4-[(3- | PD 174265 | Calbiochem, San Diego, |
| Bromophenyl)amino]-6- | | CA |
| propionylamidoquinazoline | | |
| 4-Amino-7- | PP3 | Calbiochem, San Diego, |
| phenylpyrazol[3,4- | | CA |
| d]pyrimidine | | |
| 5-Amino-N-(2,5- | Lavendustin C Methyl | Calbiochem, San Diego, |
| dihydroxybenzyl)methyl | Ester | CA |
| Salicylate | | |
| 6-Amino-4-[(3- | PD 156273 | Calbiochem, San Diego, |
| bromophenyl)amino]-7- | | CA |
| (methylamino)quinazoline | | |
| 8-[(3- | BPIQ-II | Calbiochem, San Diego, |
| Bromophenyl)amino]-1H- | | CA |
| imidazo[4,5-g]-quinazoline | | |
| 8-[(3- | BPIQ-I | Calbiochem, San Diego, |
| Bromophenyl)amino]-3- | | |
| methyl-3H-imidazo[4,5-g]- | | |
| quinazoline | | |
| 4-(3-Bromoanilino)-6,7- | AG 1517, PD 153035 | Calbiochem, San Diego, |
| dimethoxyqunazoline | | CA |
| 4-Amino-5-(4- | AG 527, PP2 | Sigma-Aldrich Co., St. |
| chlorophenyl)-7-(t- | | Louis, MO; |
| butyl)pyrazolo[3,4- | | Calbiochem, San Diego, |
| d]pyrimidine | | CA |
| | AG 537, Bis-Tyrphostin | Calbiochem, San Diego, |
| | | CA |
| 5-Amino-[(N-2,5- | Lavendustin A | Calbiochem, San Diego, |
| dihydroxybenzyl)-N′-2- | | CA |
| hydroxybenzyl]salicylic | | |
| Acid | | |
| N-[4-[(3- | CL-387, 785, EKI-785 | Calbiochem, San Diego, |
| Bromophenyl)amino]-6- | | CA |
| quinazolinyl]-2-butynamide | | |
| Hydroxy-2- | HNMPA-(AM)3 | Calbiochem, San Diego, |
| naphthalenylmethylphosphonic | | CA |
| Acid | | |
| Trisacetoxymethyl Ester | | |
| | PK1 116 | Novartis, Basel, SW |
| | p60v-src 137-157 Inhibitor | Calbiochem, San Diego, |
| | Peptide | CA |
| | Phosphatidylinositol | Kamiya Biomedical, |
| | Turnover Inhibitor, Psi- | Seattle, WA. |
| | tectorigenin | |
| | Clavilactone | |
| | ICR63 | |
| | ICR80 | |
| | XR774 | |
| 4,5-dianilinophthalimide | CGP 52411 | Sigma-Aldrich Co., St. |
| | | Louis, MO |
| | PD166285 | |
| | OSI-774, Tarceva, | OSI Pharmaceuticals, |
| | erlotinib | Uniondale, N.Y. |
| (2S,6′R)-7-chloro-2′,4,6- | Sporostatin, griseofulvin | |
| trimethoxy-6′- | | |
| methylbenzofuran-2-spiro- | | |
| 1′-cyclohex-2′-ene-3,4′- | | |
| dione | | |
| | Herbimycin A | CAS 70563-58-5 |
| | | Calbiochem, San Diego, |
| | | CA |
| | MDX-447 | Medarex, Princeton, N.J. |
| 3-Cyanoquinoline | EKB-569 | Wyeth, Madison, NJ |

In one embodiment of the present invention, the inhibitor of EGFR tyrosine kinase activity is selected from the group consisting of ZD 1839, CI-1033, OSI-774, GW 2016, EKB-569, IMC-C225, MDX-447, PKI 116, ABX-EGF, AG-82, AG-18, AG-490, AG-17, AG-213, AG-494, AG-825, AG-879, AG-1112, AG-1296, AG-1478, AG-126, RG-13022, RG-14620, and AG-555.

In particular embodiments, the inhibitors of EGFR tyrosine kinase activity effectively block the phosphorylation of EGFR.

In another embodiment, the tyrosine kinase inhibitor is effective at inhibiting the EGFR-mediated induction of NOS-2 mRNA. Even further, the tyrosine kinase inhibitor is effective at inhibiting the EGFR-mediated induction of NOS-2 protein.

According to another embodiment of the invention, a method is provided for screening additional potential agents for their ability to suppress NOS-2 induction. The method comprises incubation of a potential therapeutic agent with cells possessing EGF receptors, and in particular, with human optic nerve head astrocytes. The ability of the candidate agent to inhibit any of the following: EGFR phosphorylation, EGFR translocation, NOS-2 mRNA expression, and NOS-2 protein expression is then assessed and the efficacy of the candidate is evaluated.

In still another embodiment of the invention, a method for inhibiting an inductive, pressure-sensitive promoter, while not inhibiting NOS-2 mRNA or protein induction mediated by one or more of inflammation, pathogen or injury, is provided. Preferably, the inhibitor suppresses activation of NOS-2 gene expression by selective inhibition of the pressure-sensitive promoter region of the gene. In particular embodiments, the method comprises using an inhibitor of EGFR tyrosine kinase activity to inhibit the inductive, pressure-sensitive promoter. Even further, the inductive, pressure-sensitive promoter is the human NOS-2 promoter.

Detailed methods for screening potential agents which are selective for suppression of induction of a pressure-sensitive promoter of the responsive gene are provided by the following working examples. One skilled in the art using the disclosed principles and methods and related techniques known in the art can readily adapt these screening techniques to other genes of interest inducible by application of pressure.

The present invention also contemplates a method of treating the eye of a mammal with, or at risk of, glaucoma, comprising administering to the mammal an effective amount of an inhibitor of ligand activation of EGFR, thereby inhibiting optic nerve degeneration.

In one embodiment of the present invention, the inhibitor of ligand activation of EGFR is an EGFR antibody which serves as an EGFR antagonist.

In another embodiment, the inhibitor of ligand activation of EGFR is effective at inhibiting the EGFR-mediated induction of NOS-2 mRNA. Further, the inhibitor of ligand activation of EGFR is effective at inhibiting the EGFR-mediated induction of NOS-2 protein.

The present invention provides effective and non-invasive methods of treating glaucoma and other conditions mediated at least in part by NOS-2 expression without causing untoward and unacceptable adverse effects.

Suitable subjects for the administration of the formulation of the present invention include primates, man and other animals, particularly man and domesticated animals such as cats and dogs.

The method of the present invention, in addition to administering therapeutically effective amounts of EGFR pathway inhibitors, includes a process for treatment which involves identifying a subject in need of inhibition of expression of NOS-2. This is accomplished, e.g., by diagnosing an individual as having, or being at risk of developing, a clinically diagnosable neurodegenerative disease or condition, e.g., primary glaucoma, wherein the disease or condition is mediated at least in part by NOS-2 as described herein. The method may involve assessment of the presence or effects of excessive nitric oxide, NOS-2, elevated pressure, or P-EGFR, or the neurological symptoms or effects associated therewith related to the disease or condition in question. Preferably, the method also involves monitoring of the subject during and after the course of treatment to assess the effectiveness of the inhibition of the expression of NOS-2, or to determine the need for or appropriate modifications to, further treatment.

The monitoring of the effectiveness of the treatment can be carried out by any of the techniques disclosed for diagnosis. The schedule and manner of monitoring will vary depending on parameters such as the severity of the condition requiring treatment, and the availability and health of the subject. Preferably, monitoring is carried out at more frequent intervals the more severe the condition, and at greater, but still regular, intervals, such as semi-annually, annually, or bi-annually, for more routine monitoring.

For systemic use in treatment or prophylaxis of subjects, the compounds of the invention can be formulated as pharmaceutical or veterinary compositions. Depending on the subject to be treated, the mode of administration, and the type of treatment desired (e.g., inhibition, prevention, prophylaxis, therapy), the compounds are formulated in ways consonant with these parameters. The compositions of the present invention comprise a therapeutically or prophylactically effective dosage The EGFR pathway inhibitors of this invention are preferably used in combination with a pharmaceutically acceptable carrier.

The compositions of the present invention may be incorporated in conventional pharmaceutical formulations (e.g. injectable solutions) for use in treating humans or animals in need thereof. Pharmaceutical compositions can be administered by intraocular, periocular, subcutaneous, intravenous, or intramuscular infusion or injection, or as large volume parenteral solutions and the like. The term parenteral as used herein includes subcutaneous injections, intravenous, intramuscular, intrasternal injection, or infusion techniques.

For example, a parenteral therapeutic composition may comprise a sterile isotonic saline solution containing between 0.1 percent and 90 percent weight to volume of the EGFR pathway inhibitors.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Solid dosage forms for oral administration may include capsules, tablets, pills, powders, granules and gels. In such solid dosage forms, the active compound may be admixed with at least one inert diluent such as sucrose lactose or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

The amount of active ingredient that may be combined with the carrier materials to produce a single dosage form will vary depending upon the host treated and the particular mode of administration. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated, and the particular purpose to be achieved according to the determination of those skilled in the art.

The dosage regimen for treating a disease condition with the compounds and/or compositions of this invention is selected in accordance with a variety of factors, including the type, age, weight, sex, diet and medical condition of the patient, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed may vary widely and therefore may deviate from the dosage regimen set forth above.

The pharmaceutical compositions of the present invention are beneficially administered to a human in need thereof. However, besides being useful for human treatment, these compositions are also useful for veterinary treatment of companion animals, exotic animals and farm animals, including mammals, rodents, avians, and the like in need of such treatment. More preferred animals include horses, dogs, cats, sheep, and pigs.

For topical ocular administration the novel formulations of this invention may take the form of solutions, gels, ointments, suspensions or solid inserts, formulated so that a unit dosage comprises a therapeutically effective amount of each active component or some submultiple thereof.

Typical ophthalmologically acceptable carriers for the novel formulations are, for example, water, mixtures of water and water-miscible solvents such as lower alkanols or aralkanols, vegetable oils, polyalkylene glycols, petroleum based jelly, ethyl cellulose, ethyl oleate, carboxymethylcellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, benzalkonium chloride, methyl and propyl paraben, benzyldodecinium bromide, benzyl alcohol, phenylethanol, buffering ingredients such as sodium chloride, sodium borate, sodium acetate, or gluconate buffers, and other conventional ingredients such as sorbitan monolaurate, triethanolamine, polyoxyethylene sorbitan monopalmitylate, dioctyl sodium sulfosuccinate, monothioglycerol, thiosorbitol, ethylenediamine tetra-acetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like.

The formulation may also include a gum such as gellan gum at a concentration of 0.1% to 2% by weight so that the aqueous eyedrops gel on contact with the eye, thus providing the advantages of a solid ophthalmic insert as described in U.S. Pat. No. 4,861,760.

The pharmaceutical preparation may also be in the form of a solid insert such as one which after dispensing the drug remains essentially intact as described in U.S. Pat. Nos. 4,256,108; 4,160,452; and 4,265,874; or a bio-erodible insert that either is soluble in lacrimal fluids, or otherwise disintegrates as described in U.S. Pat. No. 4,287,175 or EPO publication 0,077,261.

In general, ophthalmic and other formulations suitable for topical administration may be formulated and administered in accordance with techniques familiar to persons skilled in the art. The finished formulations are preferably stored in opaque or brown containers to protect them from light exposure, and under an inert atmosphere. These aqueous suspensions can be packaged in preservative-free, single-dose non-reclosable containers. This permits a single dose of the medicament to be delivered to the eye as a drop or ribbon, with the container then being discarded after use. Such containers eliminate the potential for preservative-related irritation and sensitization of the corneal epithelium, as has been observed to occur particularly from ophthalmic medicaments containing mercurial preservatives. Multiple dose containers can also be used, if desired, particularly since the relatively low viscosities of the aqueous suspensions of this invention permit constant, accurate dosages to be administered dropwise to the eye as many times each day as necessary. In those suspensions where preservatives are to be included, suitable preservatives are chlorobutanol, polyquat, benzalkonium chloride, cetyl bromide, sorbic acid and the like.

In accordance with the invention the active compounds (or mixtures or salts thereof) are administered in a pharmaceutically acceptable carrier in sufficient concentration so as to deliver an effective amount of the active compound or compounds to the subject tissue. Preferably, the pharmaceutical, therapeutic solutions contain one or more of the active compounds in a concentration range of approximately 0.0001% to approximately 5%, more preferably to about 1% (weight by volume) and more preferably approximately 0.0005% to approximately 0.5%, more preferably to about 0.1% (weight by volume).

Any method of administering drugs directly to the subject tissue, such as to a mammalian eye may be employed to administer, in accordance with the present invention, the active compound or compounds to the tissue to be treated. Suitable routes of administration include systemic, such as orally or by injection, topical, periocular (e.g., subTenon's), subconjunctival, intraocular, subretinal, suprachoroidal, and retrobulbar. By the term “administering directly” is meant those general systemic drug administration modes, e.g., injection directly into the patient's blood vessels, oral administration and the like, which result in the compound or compounds being systemically available. More preferably, the active useful compound or compounds are applied topically to the eye or other tissue or are injected directly into the eye or other tissue. Particularly useful results are obtained when the compound or compounds are applied topically to the eye in an ophthalmic solution, i.e. as ocular drops.

Topical pharmaceutical preparations, for example ocular drops, gels or creams, are preferred because of ease of application, ease of dose delivery and fewer systemic side effects, such as cardiovascular hypotension.

Various preservatives may be used in the pharmaceutical preparation. Preferred preservatives include, but are not limited to, benzalkonium chloride, chlorobutanol, thimerosal, phenylmercuric acetate, and phenylmercuric nitrate.

Likewise, various preferred vehicles may be used in such ophthalmic preparation. These vehicles include, but are not limited to, polyvinyl alcohol, povidone, hydroxypropyl methyl cellulose, poloxamers, carboxymethyl cellulose and hydroxyethyl cellulose.

Tonicity adjustors may be added as needed or convenient. They include, but are not limited to, salts, particularly sodium chloride, potassium chloride etc., mannitol and glycerin, or any other suitable ophthalmically acceptable tonicity adjustor.

Various buffers and means for adjusting pH may be used so long as the resulting preparation is pharmaceutically acceptable. Accordingly, buffers include but are not limited to, acetate buffers, titrate buffers, phosphate buffers, and borate buffers. Acids or bases may be used to adjust the pH of these formulations as needed.

In a similar vein pharmaceutically acceptable antioxidants include, but are not limited to, sodium metabisulfite, sodium thiosulfate, acetylcysteine, butylated hydroxyanisole, and butylated hydroxytoluene.

The pharmaceutical solution (e.g., ocular drops) may be administered to the mammalian eye as often as necessary to effectively inhibit optic nerve injury mediated by NOS-2. In other words, the pharmaceutical solution (or other formulation) which contains the NOS-2 inhibitor as the active ingredient, is administered as often as necessary to maintain the beneficial effect of the active ingredient. Those skilled in the art will recognize that the frequency of administration depends on the precise nature of the active ingredient and its concentration in the formulation. Within these guidelines it is contemplated that the formulation of the present invention will be administered to the mammalian eye or other tissue to be treated approximately once or twice daily.

One skilled in the art will appreciate that suitable methods of administering an EGFR tyrosine kinase inhibitor, which is useful in the present inventive method, are available. Although more than one route can be used to administer a particular EGFR tyrosine kinase inhibitor, a particular route can provide a more immediate and more effective reaction than another route. Accordingly, the described routes of administration are merely exemplary and are in no way limiting.

The dose administered to an animal, particularly a human, in accordance with the present invention should be sufficient to effect the desired response in the animal over a reasonable time frame. Hence, the pharmaceutical compositions of the invention are prepared in appropriate dosage unit forms. One skilled in the art will recognize that dosage will depend upon a variety of factors, including the strength of the particular EGFR tyrosine kinase inhibitor employed, the age, species, condition or disease state, and body weight of the animal. The size of the dose also will be determined by the route, timing and frequency of administration as well as the existence, nature, and extent of any adverse side effects that might accompany the administration of a particular EGFR tyrosine kinase inhibitor and the desired physiological effect. It will be appreciated by one of ordinary skill in the art that various conditions or disease states, in particular, chronic conditions or disease states, may require prolonged treatment involving multiple administrations.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. The present inventive method will typically involve the administration of from about 1 ng/kg/day to about 100 mg/kg/day, preferably from about 15 ng/kg/day to about 50 mg/kg/day, if administered systemically. Intraocular administration typically will involve the administration of from about 0.1 ng total to about 5 mg total, preferably from about 0.5 ng total to about 1 mg total. A preferred concentration for topical administration is 0.001% to 10%.

The present inventive method also can involve the co-administration of other pharmaceutically active compounds. By “co-administration” is meant administration before, concurrently with, e.g., in combination with the EGFR tyrosine kinase activity inhibitor in the same formulation or in separate formulations, or after administration of an EGFR tyrosine kinase activity inhibitor as described above. For example, intraocular pressure lowering drugs like prostaglandin analogs and derivatives, beta adrenergic blockers, adrenergic agonists, cholinergic agonists or inhibitors of carbonic anhydrase or noncorticosteroid anti-inflammatory compounds, such as ibuprofen or flubiproben, can be co-administered. Similarly, vitamins and minerals, e.g., zinc, anti-oxidants, e.g., carotenoids (such as a xanthophyll carotenoid like zeaxanthin or lutein), and micronutrients can be co-administered. In addition, other types of inhibitors of the protein tyrosine kinase pathway, which include natural protein tyrosine kinase inhibitors like quercetin, lavendustin A, erbstatin and herbimycin A, and synthetic protein tyrosine kinase αinhibitors like tyrphostins (e.g., AG490, AG17, AG213 (RG50864), AG18, AG82, AG494, AG825, AG879, AG1112, AG1296, AG1478, AG126, RG13022, RG14620 and AG555), dihydroxy- and dimethoxybenzylidene malononitrile, analogs of lavendustin A (e.g., AG814 and AG957), quinazolines (e.g., AG1478), 4,5-dianilinophthalimides, and thiazolidinediones, can be co-administered. Genistein, or an analogue, prodrug, derivative or pharmaceutically acceptable salt thereof (see Levitzki et al., Science 267: 1782-1788 (1995); and Cunningham et al., Anti-Cancer Drug Design 7: 365-384(1992)) can be co-administered. In this regard, potentially useful derivatives of genistein include those set forth in Mazurek et al., U.S. Pat. No. 5,637,703. Neutralizing proteins to growth factors, such as a monoclonal antibody that is specific for a given growth factor, e.g., VEGF (for an example, see Aiello et al., PNAS USA 92: 10457-10461 (1995)), or phosphotyrosine (Dhar et al., Mol. Pharmacol. 37: 519-525 (1990)), can be co-administered. Other various compounds that can be co-administered include protein kinase C inhibitors (see, e.g., U.S. Pat. Nos. 5,719,175 and 5,710,145), cytokine modulators, an endothelial cell-specific inhibitor of proliferation, e.g., thrombospondins, an endothelial cell-specific inhibitory growth factor, e.g., TNFα., an anti-proliferative peptide, e.g., SPARC and prolferin-like peptides, a glutamate receptor antagonist, aminoguanidine, an angiotensin-converting, enzyme inhibitor, e.g., angiotensin 11, calcium channel blockers, .PSI.-tectorigenin, ST638, somatostatin analogues, e g., SMS 201-995, monosialoganglioside GM1, ticlopidine, neurotrophic growth factors, methyl-2,5-dihydroxycinnamate, an angiogenesis inhibitor, e.g., recombinant EPO, a sulphonylurea oral hypoglycemic agent, e.g., gliclazide (non-insulin-dependent diabetes), ST638 (Asahi et al., FEBS Letter 309: 10-14 (1992)), thalidomide, nicardipine hydrochloride, aspirin, piceatannol, staurosporine. adriamycin, epiderstatin, (+)-aeroplysinin-1, phenazocine, halomethyl ketones, anti-lipidemic agents, e.g., etofibrate, chlorpromazine and spinghosines, aldose reductase inhibitors, such as tolrestat, SPR-210, sorbinil or oxygen, and retinoic acid and analogues thereof (Burke et al., Drugs of the Future 17(2): 119-131 (1992); and Tomlinson et al., Pharmac. Ther. 54: 151-194 (1992)). Selenoindoles (2-thioindoles) and related disulfide selenides, such as those described in Dobrusin et al., U.S. Pat. No. 5,464,961, are useful protein tyrosine kinase inhibitors.

In addition to pharmaceutical formulations of one or more active ingredients, the invention may also take the form of a kit comprising one or more containers of active or other ingredients which may be accompanied by instructions for carrying out the method of the invention, e.g., relating to the effective combining of ingredients or relating to the effect of inhibition of nitric oxide production.

All publications, patents, patent applications and other references cited in this application are herein incorporated by reference in their entirety as if each individual publication, patent, patent application or other reference were specifically and individually indicated to be incorporated by reference.

Other objects and advantages of the present invention will become apparent as the detailed description of the invention proceeds.

It is to be understood that the present invention has been described in detail by way of illustration and example in order to acquaint others skilled in the art with the invention, its principles, and its practical application. Further, the specific embodiments of the present invention as set forth are not intended to be exhaustive or to limit the invention, and that many alternatives, modifications, and variations will be apparent to those skilled in the art in light of the foregoing examples and detailed description. Accordingly, this invention is intended to embrace all such alternatives, modifications, and variations that fall within the spirit and scope of the following claims.

While some of the examples and descriptions above include some conclusions about the way the invention may function, the inventors do not intend to be bound by those conclusions and functions, but put them forth only as possible explanations in light of current understanding.

### BRIEF DESCRIPTION OF EXPERIMENTAL METHODS

Immunohistochemistry of Human Optic Nerve Head Tissue

Eyes from fifteen patients with documented primary open-angle glaucoma (aged 50-92) and 12 normal (aged 52-96) were obtained within 24 hours after death from eyebanks throughout the United States. Donors had no history of neurological diseases or diabetes. Primary open angle glaucoma was defined by a clinical history of observation and treatment by an ophthalmologist and the appearance of moderate to advanced optic nerve damage on histological examination, as evidenced by the presence of a cup and the disorganization of glial columns and cribriform plates. The ONH was dissected and processed as 6 μm paraffin sagittal sections. Immunohistochemistry was performed using specific primary antibody against EGFR and p-EGFR (Santa Cruz Biotechnology Inc, Santa Cruz, Calif.) and the Vectastain Elite ABC kit (Vector Labs, Burlingame, Calif.), using diaminobenzidine as substrate. Hematoxylin was the counter stain. Double immunofluorescent labeling with p-EGFR and glial fibrillary acidic protein (GFAP)(Sigma-Aldrich Corp, St. Louis, Mo.) was as described previously.⁵ Slides were photographed using a microscope (Olympus AX70, Tokyo, Japan) equipped with a digital camera (Spot, Diagnostic Instruments Inc., Sterling Heights, Mich.).

Primary Astrocyte Culture from Human ONH Lamina cribrosa

Twelve normal human (aged 22-51) eyes were obtained within 24 hrs after death. Lamina cribrosa astrocytes were obtained as described previously.⁵ The second-passage cell cultures, which had over 95% cells positive for GFAP, were grown to 60-80% confluency and placed in serum free medium for one week before use.

Application of Elevated Hydrostatic Pressure, Cytokines and Signal Transduction Pathway Inhibitors

Astrocytes grown on coverslips were placed under sterile, disposable polypropylene cylinders 6 cm high and 3 cm in diameter. Hydrostatic pressure was elevated by 5 cm of serum free medium, conditioned at 37° C. in a 5% CO₂ humidified atmosphere. As control, the same number of cells on a coverslip was placed in 140 mm Petri dishes with 0.3 cm height of media in a volume equivalent to that used in the hydrostatic pressure experiments. Both pressurized and control cultures were maintained in a tissue culture incubator at 37° C. with a 5% CO₂ humidified atmosphere for various periods. AG82 or AG18 (Calbiochem, Darmstadt, Germany) was added to the cell media 30 min before and during the period of exposure to elevated hydrostatic pressure or IL-1β/IFN-γ at final concentrations of 7 μM or 40 μM, respectively. SB202190 (Calbiochem, Darmstadt, Germany) was added to the cell media 30 min before and during the period of exposure to elevated hydrostatic pressure or IL-1β/IFN-γ at a final concentrations of 380 nM. SN-50 (BioMol, Plymouth Meeting, Pa.) was added to the cell media 30 min before and during the period of exposure to elevated hydrostatic pressure or IL-1β/IFN-γ at a final concentrations of 50 μg/ml. Monoclonal anti-EGFR antibody (Santa Cruz Biotechnology Inc, Santa Cruz, Calif.) which recognizes an EGF receptor cell surface epitope and is an antagonist of EGFR, was added to the cell media 12 hrs before and during the period of exposure to elevated hydrostatic pressure or EGF (100 ng/ml)(Sigma-Aldrich Corp., St. Louis, Mo.) at a working dilution of 1:20. Astrocytes were stimulated by the addition to the media of 200 U/ml human IFN-γ (Sigma-Aldrich Corp, Saint Louis, Mo.) and 10 ng/ml human IL-1β (Sigma-Aldrich Corp, Saint Louis, Mo.) for 12 or 48 hours.

Immunocytochemistry

As described previously,⁵ cells grown on coverslips were fixed in 4% paraformaldehyde, incubated with specific primary antibodies against EGFR, p-EGFR or GFAP and appropriate fluorescent conjugated secondary antibodies (Molecular Probes Inc., Eugene, Ore), and visualized by fluorescence microscopy.

Western blot

NOS-2 immunoblot was as described previously.⁵ For EGFR and p-EGFR immunoblot,¹¹ astrocytes were lysed in buffer (20 mm HEPES, pH 7.0, 10 mM KCI, 2 mM MgCl₂, 0.5% Nonidet P40, 1 mM Na₃VO₄, 1 mM PMSF, 0.15 U ml⁻¹ aprotinin) and homogenized. The non-nuclear and nuclear fractions were separated by centrifugation at 1,500 g for 5 min to sediment the nuclei. The nuclear pellet was washed with the lysis buffer and resuspended in the same buffer containing 0.5 M NaCl to extract nuclear proteins. 20 μg protein of each non-nuclear fraction and 40 μg of each nuclear fraction were separated by 10% SDS-PAGE and transferred to a nitrocellulose membrane. Immunoblotting was performed with specific primary antibody against EGFR and p-EGFR followed by peroxidase-conjugated secondary antibody (Santa Cruz Biotechnology Inc, Santa Cruz, Calif.) and imaged by the enhanced chemiluminescence detection system (Amersham Life Science Inc, Arlington Heights, Ill.).

Semi-Quantitative RT-PCR

Semi-quantitative RT-PCR was performed as published previously.⁸

### EXAMPLE 1

Expression of EGFR in Human Glaucomatous Optic Nerve Heads

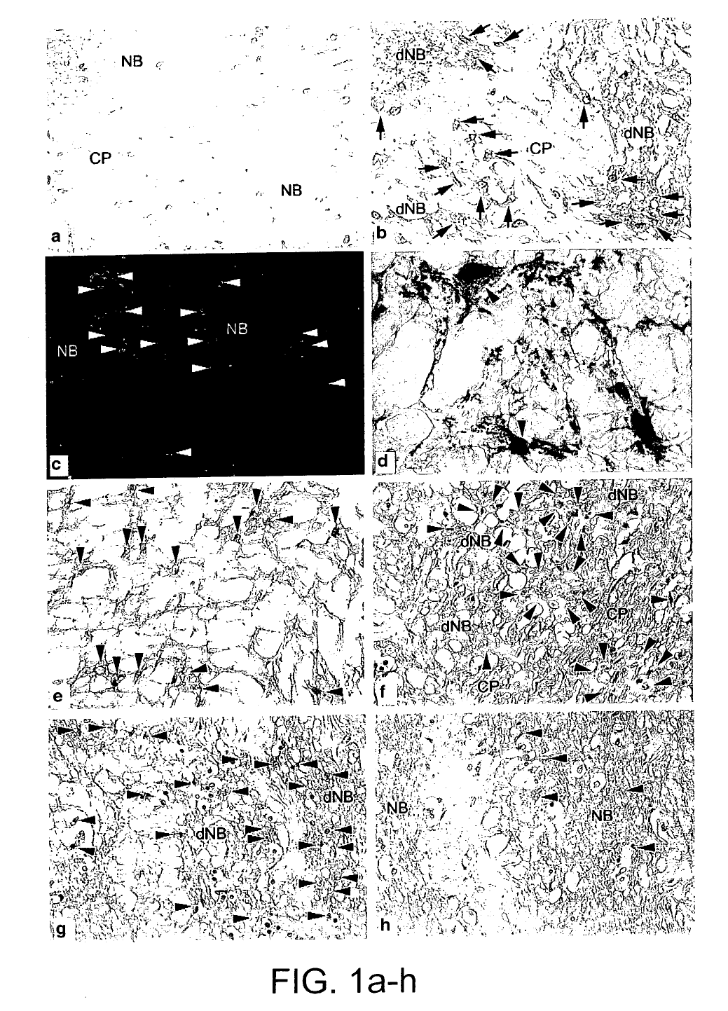

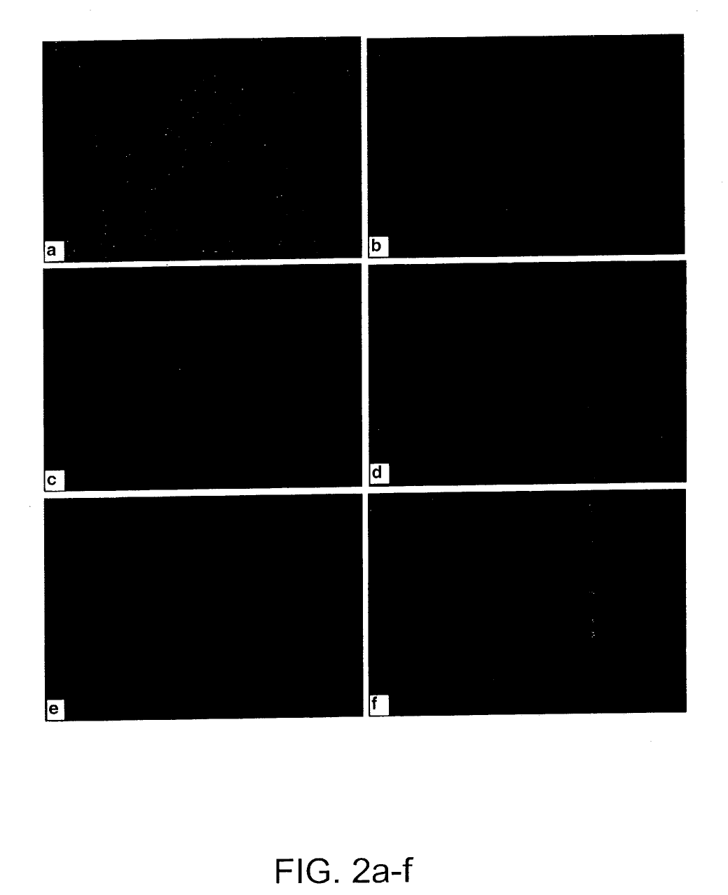

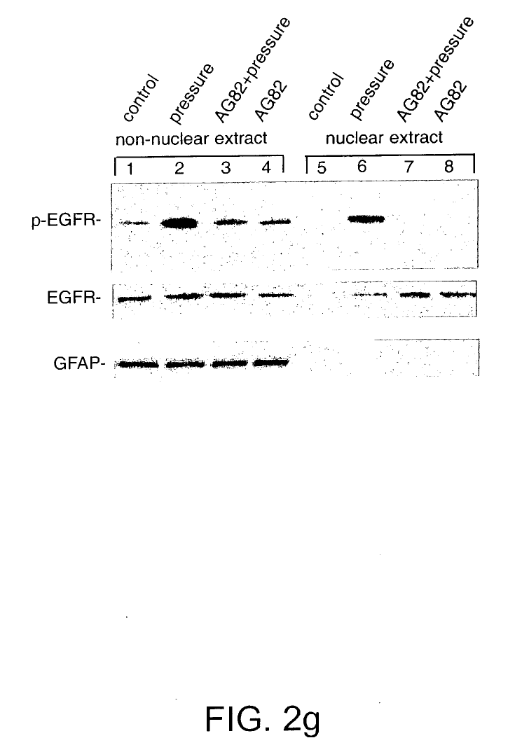

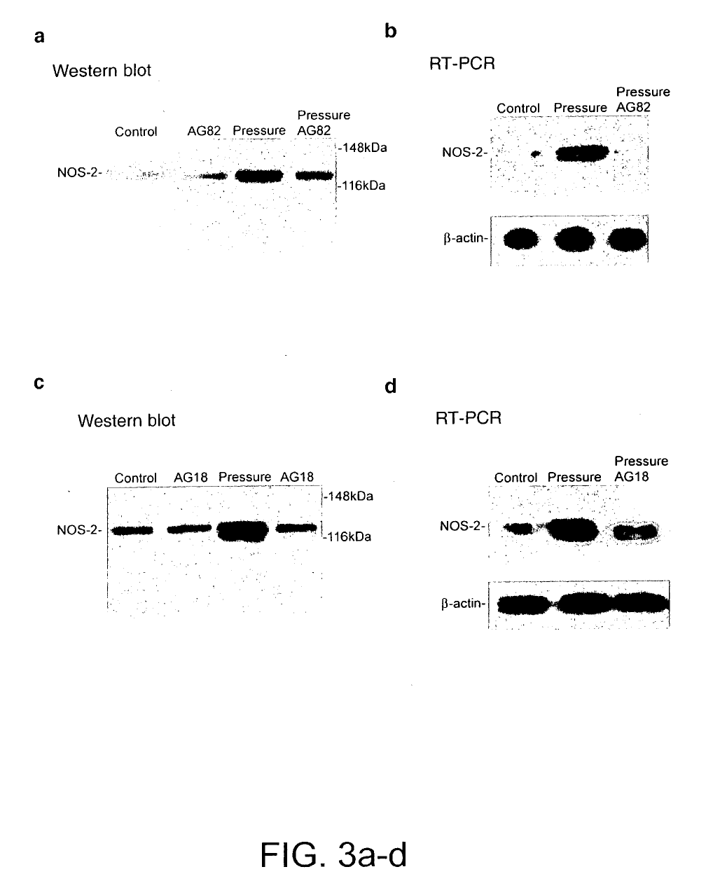

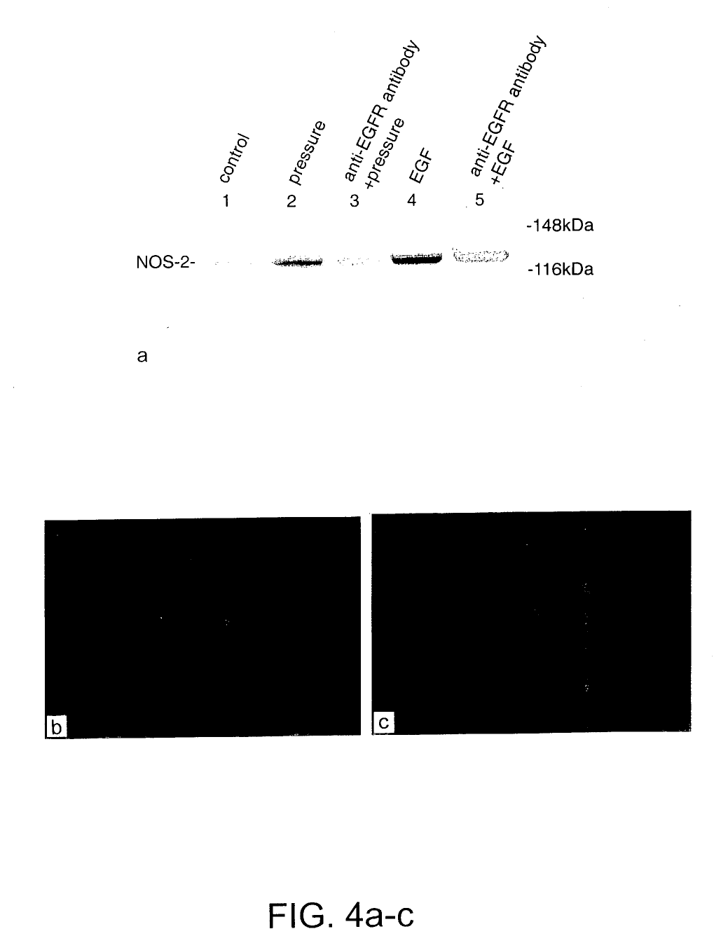

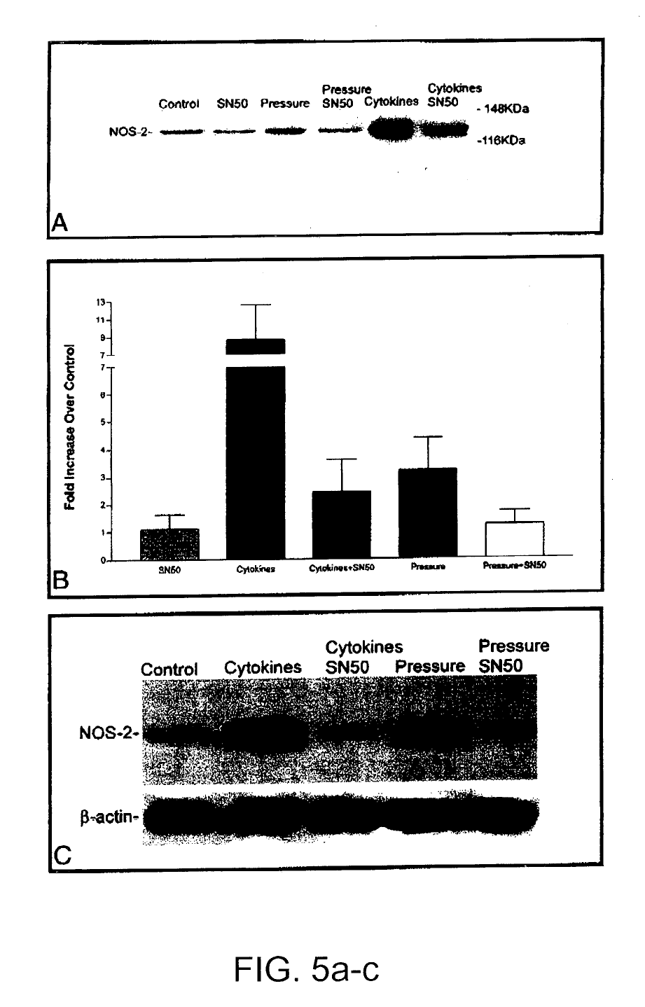

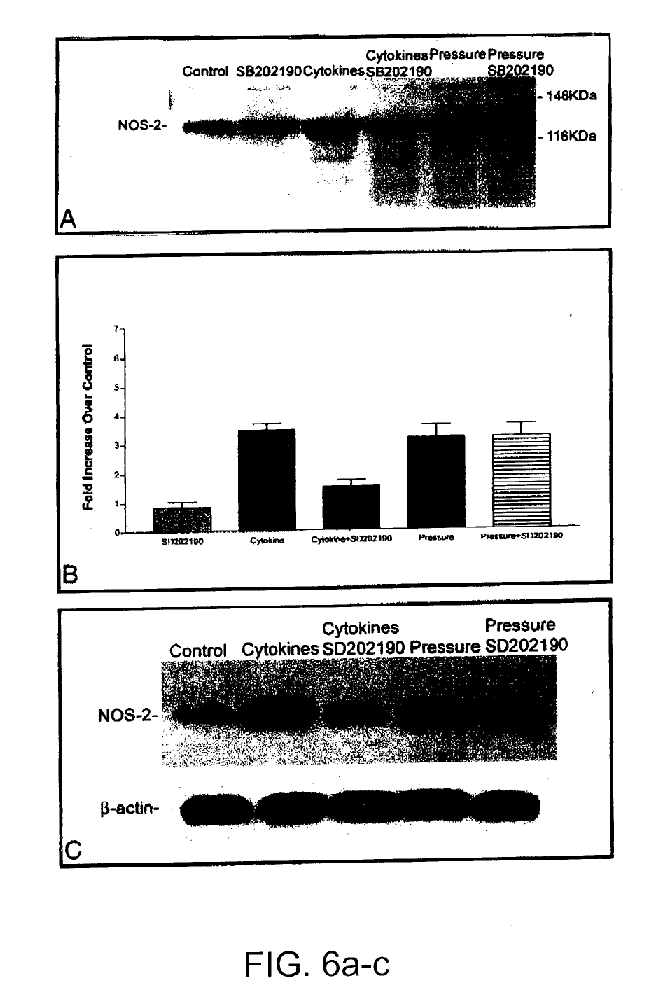

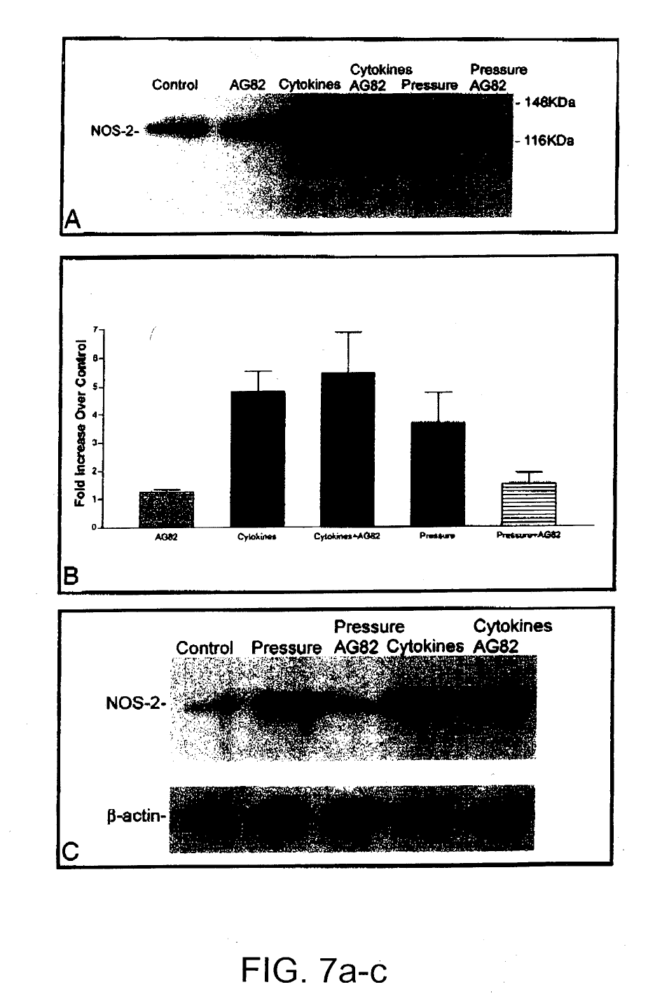

Immunohistochemistry was used to investigate the presence of EGFR in human ONHs of normal and glaucomatous eyes. In normal human ONHs, there were very few positively labeled cells for EGFR (FIG. 1a). In glaucomatous ONHs, abundant EGFR positive cells were easily identified in damaged nerve bundles throughout the prelaminar, lamina cribrosa and postlaminar region, especially in the disorganized areas of the ONH (FIG. 1b).

### EXAMPLE 2

Detection of Phosphorylated EGFR in Glaucomatous ONHs

To study the activity status of EGFR, autophosphorylated EGFR (p-EGFR) was detected by an antibody raised against the amino acid sequence containing the phosphorylated tyrosine residue (Tyr-1173) of human EGFR. Immunohistochemical labeling for p-EGFR was rarely identified in normal ONHs. In contrast, in glaucomatous ONHs, abundant immunohistochemical labeling for p-EGFR was detected throughout the prelaminar, lamina cribrosa and postlaminar regions. Double labeling of glaucomatous ONHs with GFAP demonstrated that p-EGFR was co-localized with GFAP, which indicates that the cells that contain p-EGFR are astrocytes (FIG. 1c). The immunohistochemical labeling for p-EGFR in the astrocytes of the glaucomatous ONHs was mostly located in the cytoplasm; however, nuclear location was noted as well (FIG. 1d).

The distribution of the astrocytes that contained p-EGFR in glaucomatous ONHs was apparently associated with areas where there were damaged nerve fiber bundles (FIG. 1e-h). In the prelaminar region of glaucomatous ONHs, almost all of the astrocytes in severely damaged areas were intensely labeled for p-EGFR (FIG. 1e). In the lamina cribrosa region of glaucomatous ONHs, clustered astrocytes that distributed near the remnant nerve bundles and inside the compressed cribriform plates had strong immunohistochemical labeling for p-EGFR (FIG. 1f). In the postlaminar region of glaucomatous ONH, most of the astrocytes were positive for p-EFGR in disorganized regions (FIG. 1g). However, in relatively normal appearing regions of glaucomatous ONHs, even adjacent to disorganized regions, p-EGFR positive astrocytes were much less frequent (FIG. 1h).

### EXAMPLE 3

Immunocytochemical Analysis of the Effect of Elevated Hydrostatic Pressure in Human ONH Astrocytes in vitro

To study the activity of EGFR and the regulation of its autophosphorylation in glaucomatous optic neuropathy in human optic nerve astrocytes in vitro, primary tissue cultures of lamina cribrosa astrocytes were obtained from twelve normal human eyes from donors with no history of eye disease. There was intense labeling for EGFR in second passage astrocytes of primary cultures from normal ONHs (FIG. 2a). The presence of EGFR in cultured astrocytes, but its absence in astrocytes in vivo, indicates that normal human ONH astrocytes upregulate EGFR when taken from in vivo to in vitro cell culture. The reason for astrocytes to upregulate EGFR in cell culture is unknown but may imply that these are not quiescent astrocytes. Under high magnification, the labeling for EGFR appeared granular and distributed throughout the cell body with more intense labeling in close proximity to the nucleus (FIG. 2b). Immunocytochemical labeling for p-EGFR showed a very faint presence for p-EGFR, which was evenly distributed in the cytoplasm and on the cell membrane, but not in the nucleus of the cultured normal human optic nerve astrocytes (FIG. 2c).

The primary cause of glaucoma in most patients is abnormally elevated intraocular pressure. The nature of the intraocular pressure related biomechanical stress that affects astrocytes in vivo is unknown, but sheer, tensile or compressive forces may contribute. In vitro, we used a cell culture model to test the effects of elevated hydrostatic pressure on the astrocytes of the human ONH. Primary astrocyte cultures were exposed to elevated hydrostatic pressure, produced by 10 cm depth of growth medium in a column placed over the coverslip containing cells for 10 min. The cells were then studied by immunocytochemistry for EGFR and p-EGFR.

There were no differences in the immunolabeling for EGFR between control astrocytes and those under elevated hydrostatic pressure. However, remarkably enhanced labeling for p-EGFR and especially intense labeling for p-EGFR in the nuclei of the astrocytes were clearly detected after exposure to elevated hydrostatic pressure for 10 min (FIG. 2d). The morphologic distribution of immunolabeling for p-EGFR was obviously different from the labeling for EGFR in the astrocytes exposed to elevated hydrostatic pressure, suggesting that when specific EGFRs in the astrocytes are tyrosine phosphorylated, the p-EGFR redistributes. In the cytoplasm, the immunocytochemical labeling for p-EGFR after exposure to elevated hydrostatic pressure was both granularly distributed throughout the cell body and also filamentous. The intensely labeled filaments appeared to be associated with cytoskeleton, forming a ring around the nucleus and radiating throughout the cell body and cell processes (FIG. 2e). These results demonstrate that elevated hydrostatic pressure rapidly causes tyrosine phosphorylation of EGFR.

### EXAMPLE 4

Cellular Fractionation Analysis of the Effect of Elevated Hydrostatic Pressure in Human ONH Astrocytes in vitro

To confirm the enhanced phosphorylation of EGFR in the nuclei of astrocytes in response to elevated hydrostatic pressure, we separated the cell lysates into non-nuclear and nuclear fractions prior to immunoblotting for p-EGFR and EGFR (FIG. 2g, lanes 1, 2, 5 and 6). In control astrocytes not exposed to elevated hydrostatic pressure, a weak band of p-EGFR was detected in the non-nuclear extract and there was no detectable p-EGFR in the nuclear extract. After the cells were exposed to elevated hydrostatic pressure for 10 min, the level of p-EGFR in non-nuclear extracts increased significantly. Importantly, after 10 min exposure to elevated hydrostatic pressure, the activated form of EGFR, p-EGFR, was localized in the nucleus. EGFR was detected in both non-nuclear and nuclear fractions and the quantity of EGFR appeared the same in the control astrocytes and in astrocytes exposed to elevated hydrostatic pressure. The results of the immunoblotting studies confirm our results using immunocytochemistry. Recently, nuclear localization of EGFR and its potential role as a transcription factor has been reported.¹¹

### EXAMPLE 5

Effect of Tyrosine Kinase Inhibitors on EGFR Activation in Response to Elevated Hydrostatic Pressure in Human ONH Astrocytes in vitro

We further tested the effects of tyrosine kinase inhibitors on the tyrosine phosphorylation of EGFR in response to elevated hydrostatic pressure in human ONH astrocytes. Treatment with the tyrosine kinase inhibitor AG82 blocked the increased immunocytochemical labeling for p-EGFR in the cytoplasm in response to elevated hydrostatic pressure and, most notably, there was no nuclear labeling for p-EGFR (FIG. 2f). As detectable by immunoblot, treatment with AG82 significantly blocked the tyrosine phosphorylation of EGFR in both non-nuclear and nuclear extracts of the astrocytes in response to elevated hydrostatic pressure (FIG. 2g, lanes 3, 4, 7 and 8).

### EXAMPLE 6

Effect of Tyrosine Kinase Inhibitors on the Induction of NOS-2 in Response to Elevated Hydrostatic Pressure in Human ONH Astrocytes in vitro

We have found that elevated hydrostatic pressure can induce NOS-2 expression in human ONH astrocytes in vitro.⁸ Tyrosine kinase inhibitors, which block tyrosine phosphorylation of EGFR, were tested for their effects on the induction of NOS-2 in astrocytes in response to elevated hydrostatic pressure. NOS-2 expression was studied by immunoblot for protein detection and by semi-quantitative RT-PCR for mRNA detection. When placed under a column of 5 cm depth of growth media for 48 hrs, the astrocytes were induced to synthesize abundant NOS-2 protein. AG82 significantly prevented the increased protein level of NOS-2 induced by elevated hydrostatic pressure (FIG. 3a). When placed in the column for 12 hrs, NOS-2 mRNA transcription was significantly induced and almost completely prevented by treatment with AG82 (FIG. 3b). We also tested another, more specific, EGFR tyrosine kinase inhibitor and found that AG18 also prevented both the increased protein level and the mRNA level of NOS-2 induced by elevated hydrostatic pressure (FIGS. 3c, d). These results suggest that EGFR tyrosine kinase inhibitors block the induction of NOS-2 at the gene transcriptional level and that their efficacy is based on inhibiting the tyrosine phosphorylation of EGFR in human ONH astrocytes in response to elevated hydrostatic pressure.

To further demonstrate that ligand dependent activation of EGFR regulates NOS-2 induction in astrocytes of the human ONH, we determined if EGF could induce NOS-2 expression in these cells. When 100 ng/ml EGF was added to the culture medium for 48 hrs, the protein level of NOS-2 was significantly increased in the astrocytes treated with EGF compared with non-treated astrocytes detected by Western blot (FIG. 4a, lane 4) and immunocytochemistry (FIGS. 4b, c). In addition, the morphological appearance of the astrocytes significantly changed after the cells were treated with EGF. In response to EGF, the astrocyte cell bodies became larger and elongated with very long processes. EGF treated astrocytes were intensely positive for GFAP, indicative of the reactive phenotype (FIG. 4c). Pretreatment with anti-EGFR antibody also blocked the induction of NOS-2 by EGF (FIG. 4a, lane 5), confirming the antagonistic function of this antibody. The data generated supports a role for nuclear EGFR in regulating NOS-2 gene expression. While not being bound to a particular theory, that role is believed to occur at a binding site in the promoter region of the NOS-2 gene.

### EXAMPLE 7

Effect of an NF-κB Inhibitor (SN-50) on the Induction of NOS-2 in Response to Elevated Hydrostatic Pressure and Cytokines in Human ONH Astrocytes in vitro

The immunoblot in FIG. 5a also shows the effects of the inhibitor of NF-κB, SN-50, on the appearance of NOS-2 protein in human optic nerve astrocytes treated with either cytokines or elevated hydrostatic pressure. SN-50 significantly blocked the appearance of NOS-2 protein to both cytokines and elevated hydrostatic pressure. In FIG. 5b, scans of several gels are normalized to the amount of NOS-2 protein present in the optic nerve head astrocytes under control conditions. In this set of experiments, the cell cultures used had a marked response to cytokines. Nevertheless, SN-50 significantly blocked the increased NOS-2 protein that appears 48 hours after exposure to cytokines or elevated hydrostatic pressure. To determine whether the changes in protein synthesis were due to different levels of gene transcription, mRNA was isolated and Northern blot analyses were performed. The Northern blot in FIG. 5c demonstrates that cytokines and elevated hydrostatic pressure increase gene transcription and that SN-50 affects the transcription of the NOS-2 gene to mRNA for both cytokine and elevated hydrostatic pressure stimuli.

### EXAMPLE 8

Effect of a MAP Kinase Inhibitor (SD202190)on the Induction of NOS-2 in Response to Elevated Hydrostatic Pressure and Cytokines in Human ONH Astrocytes in vitro

The immunoblot data in FIG. 6a shows the effects of the inhibitor of MAP kinase, SD202190, on the appearance of NOS-2 protein in human optic nerve astrocytes treated with either cytokines or elevated hydrostatic pressure. In FIG. 6b, scans of several gels are normalized to the amount of NOS-2 protein present in the optic nerve head astrocytes under control conditions. In this set of experiments, the cell cultures used had a smaller response to cytokines and approximately the same response to elevated hydrostatic pressure. SD202190 significantly blocked the appearance of NOS-2 protein in response to cytokine stimulation but not to elevated hydrostatic pressure. To determine whether the inhibition of NOS-2 protein synthesis in the presence of SD202190 was due to a decreased level of gene transcription, mRNA was isolated and Northern blot analyses were performed. The Northern blot in FIG. 6c demonstrates that SD202190 affects the transcription of the NOS-2 gene to mRNA in response to cytokines but not to elevated hydrostatic pressure.

### EXAMPLE 9

Effect of a Tyrosine Kinase Inhibitor (AG82) on the Induction of NOS-2 in Response to Elevated Hydrostatic Pressure and Cytokines in Human ONH Astroctyes in vitro

The immunoblot data in FIG. 7a shows the effects of the inhibitor of protein tyrosine kinase, AG82, on the appearance of NOS-2 protein in human optic nerve astrocytes treated with either cytokines or elevated hydrostatic pressure. In FIG. 7b, scans of several gels are normalized to the amount of NOS-2 protein present in the optic nerve head astrocytes under control conditions. In this set of experiments, the cell cultures used had similar responses to cytokines and to elevated hydrostatic pressure, comparable to the data shown in FIGS. 6a-c. AG82 significantly blocked the appearance of NOS-2 protein in response to elevated hydrostatic pressure but not to cytokine stimulation. To determine whether the inhibition of NOS-2 protein synthesis in the presence of AG82 was due to a decreased level of gene transcription, mRNA was isolated and Northern blot analyses were performed. The Northern blot in FIG. 7c demonstrates that AG82 affects the transcription of the NOS-2 gene to mRNA in response to elevated hydrostatic pressure but not to cytokines.

Our results confirm that several signal transduction pathways participate in the induction of the gene expression of NOS-2. In response to specific stimuli, products of the NF-κB pathway, the MAP kinase pathway and the protein tyrosine kinase pathway contribute transcription factors to activate the promoter region of the NOS-2 gene. Nevertheless, we can distinguish at least one signal transduction pathway that is common for two different stimuli and two signal transduction pathways, working in conjunction with the common pathway, that are specific for the two different stimuli. Thus, the mechanisms driven by different stimuli for activation in the promoter region of the NOS-2 gene utilize at least some different signal transduction pathways.

The transcription factor, NF-κB, contributes to the regulation of the expression of a wide variety of genes. Upon activation of this pathway, the molecule is liberated from an inhibited state in the cytoplasm and translocates to the nucleus. SN-50 inhibits the liberation of free NF-κB in the cytoplasm. The promoter region of the human NOS-2 gene contains multiple binding sites for NF-κB. Because SN-50 blocks the appearance of mRNA for NOS-2 in response both to cytokines and to elevated hydrostatic pressure, our data demonstrate that NF-κB participates in the induction of this gene in response to both stimuli.

These data confirm that several, independent signal transduction pathways participate in the induction of the gene expression of NOS-2. The results indicate that in response to specific stimuli, products of the NFκB pathway, the MAP kinase pathway and the protein tyrosine kinase pathway contribute transcription factors to activate the promotor region of the NOS-2 gene. We can distinguish at least one signal transduction pathway that is common for two different stimuli and two signal transduction pathways, working in conjunction with the common pathway, that are specific for the two different stimuli. Thus, the data indicates that the intracellular mechanisms driven by different stimuli for activation in the promotor region of the NOS-2 gene utilize at least some similar and some different signal transduction pathways.

The transcription factor, NFκB, contributes to the regulation of the expression of a wide variety of genes. Upon activation of this pathway, the molecule is liberated from an inhibited state in the cytoplasm and translocates to the nucleus. SN50 inhibits the liberation of free NFκB in the cytoplasm(30; 31). The promotor region of the human NOS-2 gene contains multiple binding sites for NFκB (32). The NFκB pathway is necessary for the induction of NOS-2 in response to IL-1β, LPS, IFNγ, and TNFα. Because SN50 blocks the appearance of mRNA for NOS-2 in response both to cytokines and to elevated hydrostatic pressure in human optic nerve astrocytes, these data demonstrate that NFκB participates in the induction of this gene in response to both stimuli.

The MAP kinase pathways have multiple forms including p38^{MAPK}, p42/44^{MAPK} and JNK. These kinases act through phosphorylation of transcription factors that translocate to the nucleus. The participation of the different MAP kinase pathways and their products in the regulation of gene expression of NOS-2 appears to be specific for cell type (33-35). The inhibitor, SD202190, is relatively selective for the p38^{MAPK} pathway (36). The above results demonstrating that this inhibitor blocks the induction of NOS-2 in human optic nerve astrocytes in response to cytokines are consistent with previous results using chondrocytes (33) and retinal pigmented epithelial cells (37) to demonstrate the participation of p38^{MAPK} in the response to cytokines. Nevertheless, the p38^{MAPK} pathway does not participate in the induction of NOS-2 in response to elevated hydrostatic pressure in human optic nerve astrocytes.

The family of signal transduction pathways known as protein tyrosine kinases also have many members. Protein tyrosine kinases mediate signals from a variety of external signaling proteins, including growth factors like EGF, FGF and TGF. Activation of these protein tyrosine kinases is by phosphorylation. In the retinal pigmented epithelium, there is a complex regulation of NOS-2 induction by FGF and TGF (38). Applicants have previously reported the presence of NOS-2 in reactive astrocytes of the optic nerve head in patients with glaucoma (39). As demonstrated above the induction of NOS-2 in human optic nerve astrocytes in response to elevated hydrostatic pressure is mediated by ligand activated EGF receptor tyrosine kinase. These results with AG82, which blocks induction of NOS-2 in response to elevated hydrostatic pressure further confirm this result. AG82 is a tyrophostin that blocks the activation by phosphorylation of the EGF receptor tyrosine kinase (40). The complex of EGF bound to the phosphorylated EGF receptor tyrosine kinase can translocate to the nucleus and act as a transcription factor (41). AG82 does not inhibit the induction of NOS-2 in response to cytokines, indicating that activated EGF receptor tyrosine kinase does not participate in the cytokine response.

Although the excessive NO produced by NOS-2 is cytodestructive, the functional results of a cell inducing NOS-2 can be either protective or destructive. Cells such as macrophages induce NOS-2 to kill pathogens that can damage tissues. Cells such as glia in the CNS induce NOS-2 as part of an activation response mechanism that can inadvertently damage nervous tissue. Accordingly, applicants′ discoveries provide for selective pharmacological inhibition of signal transduction pathways that participate in the induction of NOS-2 in response to specific stimuli, but are not involved in the induction of NOS-2 in response to inflammatory stimuli, which is useful for the treatment of certain human diseases while leaving inflammatory responses intact.

NOS-2 has been implicated as participating in several neurodegenerative diseases, such as stroke,²¹ Parkinson's disease,²² Alzheimer's disease²³ and multiple sclerosis.²⁴ Despite previous reports that the level of EGFR increases in some neurodegenerative diseases,²⁵⁻²⁹ identification of EGFR as an intracellular signaling pathway mediating the induction of NOS-2, which results in neurotoxic effects, has never been suggested. Accordingly, applicants discoveries help provide for methods for pharmacological neuroprotection therapy in glaucoma and other neurodegenerative diseases via manipulation of the EGFR pathway and its role in NOS-2 synthesis.

References

1. Quigley, H. & Anderson, D. R. Invest Ophthalmol 15, 606-616 (1976).

2. Quigley, H. A., Hohman, R. M., Addicks, E. M., Massof, R. W. & Green, W. R. Am J Ophthalmol 95,673-911 (1983).

3. Hernandez, M. R., Andrzejewska, W. M. & Neufeld, A. H. Am J Ophthalmol 109,180-188 (1990).

4. Varela, H. J. & Hernandez, M. R. J Glaucoma 6, 303-313 (1997).

5. Liu, B. & Neufeld, A. H. Glia 30,178-186 (2000).

6. Neufeld, A. H., Hernandez, M. R. & Gonzalez, M. Arch Ophthalmol 115, 497-503 (1997).

7. Neufeld, A. H., Sawada, A. & Becker, B. Proc Natl Acad. Sci USA 96, 9944-9948 (1999).

8. Liu, B. & Neufeld, A. H. Arch Ophthalmol 119, 240-245 (2001).

9. Prenzel, N., Fischer, O. M., Streit, S., Hart, S. & Ullrich, A. Endocr Relat Cancer 8, 11-31 (2001).

10. Wiley, H. S. & Burke, P. M. Traffic 2,12-18 (2001).

11. Lin, S. Y., Makino, K., Xia, W., et al. Nat Cell Biol 3, 802-808 (2001).

12. Nickoloff, B. J., Mitra, R. S. J. Invest. Dermatol. 93, 799-803 (1989).

13. Imamoto, A., L. M. Beltran, J. DiGiovanni. Carcinogenesis 11,1543-1549 1990).

14. Hori, T., S. Kashiyama, M. Hayakawa, S. Shimbamoto, M. Tsujimoto, N. Oku, F. Ito. J. Cell. Physiol. 141, 275-280 (1989).

15. Murthy, U., D. J. Rieman, U. Rodeck. Biochem. Biphys. Res. Comm. 172, 471-476 (1990).

16. Rodeck, U., N. Williams, U. Murthy, M. Herlyn. J. Cell. Biochem. 44, 69-79 (1990).

17. Ennis, B. W., E. M. Valverius, S. E. Bates, M. E. Lippman, F. Bellot, R. Kris, J. Schlessinger, H. Masui, A. Goldenberg, J. Mendelsohn, R. B. Dickson. Molec. Endocrinol. 3, 1830-1838 (1989).

18. Elizalde, P. V., E. H. Charreau. Cancer Investig. 8, 365-374 (1990).

19. Matsunami, R. K., Campion, S. R., Niyogi, S. K., Stevens, A. FEBS Letters 264,105-108 (1990).

20. Eppstein, D. A., Marsh, Y. V., Schryver, B. B., Bertics, P. J. J. Cell. Physiol. 141, 420-430 (1989).

21. Love, S. Brain Pathol9, 119-131 (1999).

22. Liberatore G. T., et al. Nat Med 5, 1403-1409 (1999).

23. Luth H. J. and Arendt T. J. Himforsch. 39, 245-251 (1998).

24. Lui J. S., Zhao M. L., Brosnan C. F., Lee S. C. Am J Pathol 158, 2057-2066 (2001).

25. Nieto-Sampedro, M., Gomez-Pinilla, F., Knauer, D. J. & Broderick, J. T. Neurosci Left 91, 276-282 (1988).

26. Planas, A. M., Justicia, C., Soriano, M. A. & Ferrer, I. Glia 23,120-129 (1998).

27. Werner, M. H., Nanney, L. B., Stoscheck, C. M. & King, L. E. J Histochem Cytochem 36, 81-86 (1988).

28. Villares, J., Faucheux, B., Herrero, M. T., et al. Exp Neurol 154, 146-156 (1998).

29. Ferrer, I., Alcantara, S., Ballabriga, J., et al. Prog Neurobiol 49, 99-123 (1996).

30. Cheng, N., Shi, X., Ye, J., Castranova, V., Chen, F., Leonard, S. S., Vallyathan, V., and Rojanasakul, Y. (1999). Role of transcription factor NF-β in asbestos-induced TNFα response from macrophages. Exp. Mol. Pathol. 66, 201-210.

31. Panet, H., Brazilai, A., Daily, D., Melamed, E., and Offen, D. (2001). Activation of nuclear transcription factor β (NF-β) is essential for dopamine-induced apoptosis in PC12 cells. J. Neurochem. 77, 391-398.

32. Taylor, B. S., de Vera, M. E., Ganster, R. W., Wang, Q., Shapiro, R. A., Morris, S. M., Jr., Billiar, T. R., and Geller, D. A. (1998). Multiple NF-β enhancer lements regulate cytokine induction of the human inducible nitric oxide synthase gene. J. Biol. Chem. 273, 15148-15156.

33. Mendes, A. F., Caramona, M. M., Carvalho, A. P., and Lopes, M. C. (2002). Role of mitogen-activated protein kinases and tyrosine kinases on IL-1-Induced NF-β activation and iNOS expression in bovine articular chondrocytes. Nitric. Oxide, 6, 35-44.

34. Cruz, M. T., Durate, C. B., Goncalo, M., Carvalho, A. P., and Lopes, M. C. (1999). Involvement of JAK2 and MAPK on type II nitric oxide synthase expression in skin-derived dendritic cells. Am. J. Physiol, 277, C1050-C1057.

35. Kristof, A. S., Marks-Konczalik, J., and Moss, J. (2001). Mitogen-activated protein kinases mediate activator protein-1-dependent human inducible nitric-oxide synthase promoter activation. J. Biol. Chem., 276, 8445-8452.

36. Ajizian, S. J., English, B. K., and Meals, E. A. (1999). Specific inhibitors of p38 and extracellular signal-regulated kinase mitogen-activated protein kinase pathways block inducible nitric oxide synthase and tumor necrosis factor accumulation in murine macrophages stimulated with lipopolysaccharide and interferon-gamma. J. Infect. Dis., 179, 939-944.

37. Faure, V., Hecquet, C., Coutois, Y., and Goureau, O. (1999). Role of interferon regulatory factor-1 and mitogen-activated protein kinase pathways in the induction of nitric oxide synthase-2 in retinal pigmented epithelial cells. J. Biol. Chem., 274, 4794-4800.

38. Faure, V., Courtois, Y., and Goureau, O. (1999). Differential regulation of nitric oxide synthase-II mRNA by growth factors in rat, bovine, and human retinal pigmented epithelial cells. Ocul. Immunol. Inflamm., 7, 27-34.

39. Liu, B. and Neufeld, A. H. (2000). Expression of nitric oxide synthase-2 (NOS-2) in reactive astrocytes of the human glaucomatous optic nerve head. Glia, 30, 178-186.

40. Tsuda, T., Kusui, T., and Jensen, R. T. (1997). Neuromedin B receptor activation causes tyrosine phosphorylation of p125FAK by a phospholipase C independent mechanism which requires p21rho and integrity of the actin cytoskelton. Biochemistry, 36, 16328-16337.

41. Lin, S. Y., Makino, k., Xia, W., Matin, A., Wen, Y., Kwong, K. Y., Bourguignon, L., and Hung, M. C. (2001). Nuclear localization of EGF receptor and its potential new role as a transcription factor. Nat. Cell Biol., 3, 802-808.

What is claimed is:

1. A method of inhibiting expression of NOS-2 in a subject in need of such inhibition, comprising administering to the subject an effective amount of an inhibitor of the EGFR pathway.

2. The method of claim 1 wherein the inhibitor is a tyrosine kinase inhibitor or an inhibitor which directly or indirectly inhibits EGFR.

3. The method of claim 1 wherein the inhibitor treats or prevents a condition mediated at least in part by the expression of NOS-2.

4. The method of claim 1 wherein the inhibitor treats or prevents a condition mediated at least in part by a pressure-sensitive inducer of NOS-2 expression.

5. The method of claim 1 wherein the method further comprises inhibiting translocation of EGFR or P-EGFR.

6. The method of claim 1 wherein the effective amount is administered orally, transdermally, topically, parenterally, rectally, ophthalmically, or aurally.

7. The method of claim 6 wherein the parenteral administration is intramuscular, intravenous or subcutaneous.

8. The method of claim 3 wherein the condition is associated with a neurological disorder or neurodegenerative disease.

9. The method of claim 8 wherein the disorder or disease is selected from the group consisting of Alzheimer's disease, Parkinson's disease, amyotrophic lateral sclerosis (Lou Gehrig's disease), multiple sclerosis, motor-neuron disease, diabetic retinopathy, glaucomatous optic neuropathy, ocular hypertension, myasthenia gravis, tardive dyskinesia, dementia associated with Down's syndrome, stroke, cerebral ischemia, senile cognitive decline, a demyelinating condition or mechanical injury.

10. The method of claim 2 wherein the condition is selected from the group consisting of a degenerative bone disease, inflammatory disease, or a condition caused by compression of a tissue leading to damage, such as arthritis, osteoarthritis, and rheumatoid arthritis

11. The method of claim 1 wherein the subject is a mammal.

12. The method of claim 11 wherein the subject is a human.

13. The method of claim 2 wherein the inhibitor of EGFR prevents activation of EGFR by a ligand or ligand independent activation.

14. The method of claim 2 wherein the tyrosine kinase inhibitor is selected from the group consisting of ZD 1839, CI-1033, OSI-774, GW 2016, EKB-569, IMC-C225, MDX-447, PKI 116, ABX-EGF, AG-82, AG-18, AG-490, AG-17, AG-213, AG-494, AG-825, AG-879, AG-1112, AG-1296, AG-1478, AG-126, RG-13022, RG-14620, and AG-555.

15. A method of inhibiting the activity of a pressure-sensitive promoter in a subject through the administration of an inhibitor selected from the group consisting of a tyrosine kinase inhibitor or an inhibitor of activation of EGFR, wherein the inhibitor does not substantially inhibit the promoter's activity leading to mRNA or protein expression induced by one or more of inflammation, pathogen or injury.

16. The method of claim 15 wherein the pressure-sensitive promoter is the human NOS-2 promoter.

17. The method of claim 15 wherein the inhibitor does not substantially inhibit the promoter's activity leading to mRNA or protein expression induced by inflammation.

18. A method for treating or preventing damage associated with glaucoma comprising administering to a subject in need thereof, an effective amount of an inhibitor of NOS-2 expression to an eye of the subject.

19. The method of claim 18 wherein the inhibitor is a tyrosine kinase inhibitor or an inhibitor of ligand activation of EGFR.

20. The method of claim 18 wherein the inhibitor treats or prevents damage mediated at least in part by the expression of NOS-2.

21. The method of claim 18 wherein the inhibitor treats or prevents a condition mediated at least in part by a pressure-sensitive inducer of NOS-2 expression.

22. The method of claim 18 further comprising the inhibition of activation or translocation of EGFR or P-EGFR.

23. The method of claims 18 in which the effective amount is administered orally, transdermally, topically, parenterally, rectally, opthalmically, or aurally.

24. The method of claim 23 in which the parenteral administration is intramuscular, intravenous, or subcutaneous.

25. The method of claims 18 wherein the subject is a mammal.

26. The method of claim 25 wherein the subject is a human.

27. The method of claim 19 wherein the inhibitor of EGFR prevents activation of EGFR by a ligand.

28. The method of claim 19 wherein the tyrosine kinase inhibitor is selected from the group consisting of ZD 1839, CI-1033, OSI-774, GW 2016, EKB-569, IMC-C225, MDX-447, PKI 116, ABX-EGF, AG-82, AG-18, AG-490, AG-17, AG-213, AG-494, AG-825, AG-879, AG-1112, AG-1296, AG-1478, AG-126, RG-13022, RG-14620, and AG-555.

29. The method of claim 18 wherein the method further comprises inhibiting a pressure-sensitive promoter but wherein the inhibitor does not substantially inhibit the promoter's activity leading to mRNA or protein expression induced by one or more of inflammation, pathogen, or injury.

30. The method of claim 29 wherein the pressure-sensitive promoter is the human NOS-2 promoter.

31. The method of claim 29 wherein the inhibitor does not substantially inhibit the promoter's activity leading to mRNA or protein expression induced by inflammation.

32. A pharmaceutical composition comprising in dosage unit form an amount of an inhibitor of the EGFR pathway or a precursor, prodrug, metabolite or derivative thereof, effective to inhibit expression of NOS-2 and a pharmaceutically acceptable carrier.

33. The pharmaceutical composition of claim 32 wherein the inhibitor is a tyrosine kinase inhibitor or an EGFR antibody.

34. The pharmaceutical composition of claim 32 wherein the amount is effective to treat or prevent a condition mediated at least in part by the expression of NOS-2.

35. The pharmaceutical composition of claim 34 wherein the inhibitor treats or prevents a condition mediated at least in part by a pressure-sensitive inducer of NOS-2 expression.

36. The pharmaceutical composition of claim 32 wherein the inhibitor inhibits activation or translocation of EGFR or P-EGFR.

37. The pharmaceutical composition of claim 32 wherein the amount is effective to treat or prevent a condition which is associated with a neurological disorder or neurodegenerative disease.

38. The pharmaceutical composition of claim 37 wherein the disorder or disease is selected from the group consisting of Alzheimer's disease, Parkinson's disease, amyotrophic lateral sclerosis (Lou Gehrig's disease), multiple sclerosis, motor-neuron disease, diabetic retinopathy, glaucomatous optic neuropathy, ocular hypertension, myasthenia gravis, tardive dyskinesia, dementia associated with Down's syndrome, stroke, cerebral ischemia, senile cognitive decline, a demyelinating condition or mechanical injury.

39. The pharmaceutical composition of claim 37 wherein the pressure-sensitive condition is selected from the group consisting of a degenerative bone disease, inflammatory disease, a disease caused by compression of a tissue leading to damage, such as arthritis, osteoarthritis, and rheumatoid arthritis.

40. The pharmaceutical composition of claim 33 wherein the inhibitor of EGFR prevents activation of EGFR by a ligand.

41. The pharmaceutical composition of claim 33 wherein the tyrosine kinase inhibitor is selected from the group consisting of ZD 1839, CI-1 033, OSI-774, GW 2016, EKB-569, IMC-C225, MDX-447, PKI 116, ABX-EGF, AG-82, AG-18, AG-490, AG-17, AG-213, AG-494, AG-825, AG-879, AG-1112, AG-1296, AG-1478, AG-126, RG-13022, RG-14620, and AG-555.

42. The pharmaceutical composition of claim 33 wherein the tyrosine kinase inhibitor or EGFR antibody inhibits the activity of a pressure-sensitive promoter, but does not substantially inhibit the promoter's activity leading to mRNA or protein expression induced by one or more of inflammation, pathogen or injury.

43. The pharmaceutical composition of claim 42 wherein the pressure-sensitive promoter is the human NOS-2 promoter.

44. The pharmaceutical composition of claim 43 wherein the inhibitor does not substantially inhibit the promoter's activity leading to mRNA or protein expression induced by inflammation.

45. The pharmaceutical composition of claim 32 wherein the amount is effective for treating or preventing damage associated with glaucoma in an eye of a subject in need thereof.
